(19) United States
(12) Patent Application Publication
(10) Pub. No.: US 2015/0216973 A1
(43) Pub. Date: Aug. 6, 2015

(54) METHOD FOR THE INDUCTION OF AN IMMUNE RESPONSE

(71) Applicant: Network Immunology Inc.., Vancouver (CA)

(72) Inventors: Geoffrey W. Hoffmann, Vancouver (CA); Sybille Muller, Vancouver (CA)

(21) Appl. No.: 14/420,604

(22) PCT Filed: Aug. 8, 2013
(86) PCT No.: PCT/CA2013/000710

§ 371 Date: Feb. 9, 2015

Related U.S. Application Data
(60) Provisional application No. 61/681,040, filed on Aug. 8, 2012.
(60) Provisional application No. 61/727,539, filed on Nov. 16, 2012.
(60) Provisional application No. 61/801,153, filed on Mar. 15, 2013.

(51) Int. Cl. A61K 39/395; A61K 39/42; C07K 19/00

(52) U.S. Cl. CPC A61K 39/39566; C07K 19/00; A61K 39/42

(57) ABSTRACT

An immune response against an antigen is induced by stimulation with a two-component vaccine that includes an antiidiotypic antibody as one of the components. An immune response against an antigen is also induced by stimulation with other two-component vaccines. Stimulation by a two-component vaccine may be followed by stimulation with a monoclonal broadly neutralizing anti-HIV antibody or with one of the components of the two-component vaccine. This method of inducing immunity has applications in vaccines against infectious pathogens and in therapeutic and preventive vaccines against cancers.

### FIELD

The present disclosure relates to the modification of an immune system. At least in part the present disclosure relates to the use of a two-component vaccine to modify an immune system. Embodiments of the present disclosure include vaccines that are protective against infectious pathogens and vaccines for the treatment and prevention of cancers. The disclosure further relates to methods, pharmaceutical compositions, uses, kits, and the like.

### BACKGROUND

There are many diseases caused by infectious agents for which no vaccine is currently available or for which the current vaccine is less than optimal. Vaccines for such infectious agents would be useful.

Certain cancers are associated with the expression of antigens that may be used as markers of cancers. Vaccines that are effective in targeting these markers would be useful.

### SUMMARY

The present disclosure relates, at least in part, to a method of inducing an immune response. The method may provide for prevention or treatment of an infection by an infectious agent. The method may be useful in the prevention and treatment of cancers. The present method may comprise introducing a two-component proteomic stimulus to an immune system, the stimulus comprising an antigen “Ag” complexed to, and/or mixed with, antiidiotypic antibodies “Ab2” that are specific for antibodies “Ab1” that are specific for the antigen. The antigen Ag may, for example, be one or more components of an infectious pathogen. The antigen Ag may also be molecules associated with cancer cells, preferably the molecules are upregulated in cancer cells and/or not present on normal cells. Ab2 may also be a fragment of an antiidiotypic antibody, for example a Fab fragment.

The present disclosure relates, at least in part, to the induction of an immune response using a two-component vaccine comprising mixtures or complexes of (a) a monoclonal antiidiotypic antibody “Ab2” that is specific for antibodies “Ab1” that in turn are specific for pathogen or cancer antigens and (b) a monoclonal broadly neutralizing anti-HIV antibody such as a broadly neutralizing anti-HIV antibody (BnAb).

The present monoclonal antiidiotypic antibody Ab2 may, for example, be the IgM/κ antibody 1F7.

The present disclosure further relates, at least in part, to a method of inducing an immune response comprising immunization with a two-component vaccine followed by immunization with one of the two components of said vaccine or with a monoclonal broadly neutralizing anti-HIV antibody (BnAb).

This disclosure further relates to pharmaceutical compositions, uses and kits.

### BRIEF DESCRIPTION OF THE DRAWINGS

FIG. 1 Shows model that explains the “Oudin-Cazenave enigma”.

FIG. 2 shows possible mechanism for the induction of a strong immune response against an antigen Ag using complexes or mixtures of antiidiotypic antibodies Ab2 and the antigen Ag.

FIG. 3 shows ELISA assay results showing the immune response to gp120 and gp41 in rabbit 1, which was immunized with complexes of the monoclonal antibody 1F7 and gp120.

FIG. 4 shows ELISA assay results showing the immune response to gp120 and gp41 in rabbit 2, which was likewise immunized with complexes of the monoclonal antibody 1F7 and gp120.

FIG. 5 shows ELISA assay results showing the immune response to gp120 and gp41 in rabbit 3, which was immunized with complexes of the monoclonal antibody 1F7 and MPER.

FIG. 6 shows ELISA assay results showing the immune response to gp120 and gp41 in rabbit 4, which was likewise immunized with complexes of the monoclonal antibody 1F7 and MPER.

FIG. 7 shows ELISA assay results showing the immune response to gp120 and gp41 in rabbit 5, which was immunized with gp120.

FIG. 8 shows ELISA assay results showing the immune response to gp120 and gp41 in rabbit 6, which was immunized with a mixture of 1F7 and the monoclonal broadly neutralizing anti-HIV antibody B12.

FIG. 9 shows ELISA assay results showing the immune response to gp120 and gp41 in rabbit 7, which was immunized with 1F7 that had been sham treated, that is the step of forming complexes with gp120 or MPER were taken, but in the absence of gp120 or MPER.

FIG. 10 shows ELISA assay results showing the immune response to gp120 and gp41 in rabbit 8, which was likewise immunized with 1F7 that had been sham treated.

FIG. 11 shows ELISA assay results showing the immune response to gp120 and gp41 in rabbit 9, which was immunized with complexes of the monoclonal antibody TEPC183 and MPER.

FIG. 12 shows ELISA assay results showing the immune response to gp120 and gp41 in rabbit 10, which was immunized with complexes of the monoclonal antibody TEPC183 and MPER.

### DETAILED DESCRIPTION

The vaccines described herein may be for reducing or preventing infection with any of a large number of infectious agents. For a given infectious agent two-component vaccines comprising complexes and/or mixtures of a monoclonal antiidiotypic antibody Ab2 and an antigen or antigens Ag of the infectious agent may be used for immunization, whereby the antiidiotypic antibody Ab2 is specific for antibodies Ab1 that are specific for the pathogen. Mixtures or complexes of a monoclonal antiidiotypic antibody Ab2 and a monoclonal broadly neutralizing anti-HIV antibody may also be used for immunization. Immunization with a two-component vaccine may, for example, be followed by immunization with a BnAb or with one of the components of the two-component vaccine.

Any suitable infection may be prevented, treated, or the prevention or treatment of the disease may be aided, with the present method of inducing an immune response. A non-limiting list of certain infectious diseases and their causative agents is as follows:

TABLE 1

| Disease | Source of Disease |
|---|---|
| Acinetobacter infections | Acinetobacter baumannii |
| Actinomycosis | Actinomyces israelii, Actinomyces gerencseriae and |
| | Propionibacterium propionicus |
| African sleeping sickness (African | Trypanosoma brucei |
| trypanosomiasis) | |
| AIDS (Acquired immune deficiency | HIV (Human immunodeficiency virus) |
| syndrome) | |
| Amebiasis | Entamoeba histolytica |
| Anaplasmosis | Anaplasma genus |
| Anthrax | Bacillus anthracis |
| Arcanobacterium haemolyticum infection | Arcanobacterium haemolyticum |
| Argentine hemorrhagic fever | Junin virus |
| Ascariasis | Ascaris lumbricoides |
| Aspergillosis | Aspergillus genus |
| Astrovirus infection | Astroviridae family |
| Babesiosis | Babesia genus |
| Bacillus cereus infection | Bacillus cereus |
| Bacterial pneumonia | multiple bacteria |
| Bacterial vaginosis (BV) | multiple bacteria |
| Bacteroides infection | Bacteroides genus |
| Balantidiasis | Balantidium coli |
| Baylisascaris infection | Baylisascaris genus |
| BK virus infection | BK virus |
| Black piedra | Piedraia hortae |
| Blastocystis hominis infection | Blastocystis hominis |
| Blastomycosis | Blastomyces dermatitidis |
| Bolivian hemorrhagic fever | Machupo virus |
| Borrelia infection | Borrelia genus |
| Botulism (and Infant botulism) | Clostridium botulinum; Note: Botulism is not |
| | an infection by Clostridium botulinum but cause |
| | by the intake of botulinum toxin. |
| Brazilian hemorrhagic fever | Sabia |
| Brucellosis | Brucella genus |
| Burkholderia infection | usually Burkholderia cepacia and other Burkholderia species |
| Buruli ulcer | Mycobacterium ulcerans |
| Calicivirus infection | Caliciviridae family |
| (Norovirus and Sapovirus) | |
| Campylobacteriosis | Campylobacter genus |
| Candidiasis (Moniliasis; Thrush) | usually Candida albicans and other Candida species |
| Cat-scratch disease | Bartonella henselae |
| Cellulitis | usually Group A Streptococcus and Staphylococcus |
| Chagas Disease (American | Trypanosoma cruzi |
| trypanosomiasis) | |
| Chancroid | Haemophilus ducreyi |
| Chickenpox | Varicella zoster virus (VZV) |
| Chlamydia | Chlamydia trachomatis |
| Chlamydophila pneumoniae infection | Chlamydophila pneumoniae |
| Cholera | Vibrio cholerae |
| Chromoblastomycosis | usually Fonsecaea pedrosoi |
| Clonorchiasis | Clonorchis sinensis |
| Clostridium difficile infection | Clostridium difficile |
| Coccidioidomycosis | Coccidioides immitis and Coccidioides posadasii |
| Colorado tick fever (CTF) | Colorado tick fever virus (CTFV) |
| Common cold (Acute viral | usually Rhinoviruses and coronaviruses. |
| rhinopharyngitis; Acute coryza) | |
| Creutzfeldt-Jakob disease (CJD) | CJD prion |
| Crimean-Congo hemorrhagic fever | Crimean-Congo hemorrhagic fever virus |
| (CCHF) | |
| Cryptococcosis | Cryptococcus neoformans |
| Cryptosporidiosis | Cryptosporidium genus |
| Cutaneous larva migrans (CLM) | usually Ancylostoma braziliense; multiple other parasites |
| Cyclosporiasis | Cyclospora cayetanensis |
| Cysticercosis | Taenia solium |
| Cytomegalovirus infection | Cytomegalovirus |
| Dengue fever | Dengue viruses (DEN-1, DEN-2, DEN-3 and DEN-4) - |
| | Flaviviruses |
| Dientamoebiasis | Dientamoeba fragilis |
| Diphtheria | Corynebacterium diphtheriae |
| Diphyllobothriasis | Diphyllobothrium |
| Dracunculiasis | Dracunculus medinensis |
| Ebola hemorrhagic fever | Ebolavirus (EBOV) |
| Echinococcosis | Echinococcus genus |
| Ehrlichiosis | Ehrlichia genus |
| Enterobiasis (Pinworm infection) | Enterobius vermicularis |
| Enterococcus infection | Enterococcus genus |
| Enterovirus infection | Enterovirus genus |
| Epidemic typhus | Rickettsia prowazekii |
| Erythema infectiosum (Fifth disease) | Parvovirus B19 |
| Exanthem subitum (sixth disease) | Human herpesvirus 6 (HHV-6) and Human herpesvirus |
| | 7 (HHV-7) |
| Fasciolopsiasis | Fasciolopsis buski |
| Fasciolosis | Fasciola hepatica and Fasciola gigantica |
| Fatal familial insomnia (FFI) | FFI prion |
| Filariasis | Filarioidea superfamily |
| Food poisoning by Clostridium | Clostridium perfringens |
| perfringens | |
| Free-living amebic infection | multiple |
| Fusobacterium infection | Fusobacterium genus |
| Gas gangrene (Clostridial myonecrosis) | usually Clostridium perfringens; other Clostridium |
| | species |
| Geotrichosis | Geotrichum candidum |
| Gerstmann-Sträussler-Scheinker | GSS prion |
| syndrome (GSS) | |
| Giardiasis | Giardia intestinalis |
| Glanders | Burkholderia mallei |
| Gnathostomiasis | Gnathostoma spinigerum and Gnathostoma hispidum |
| Gonorrhea | Neisseria gonorrhoeae |
| Granuloma inguinale (Donovanosis) | Klebsiella granulomatis |
| Group A streptococcal infection | Streptococcus pyogenes |
| Group B streptococcal infection | Streptococcus agalactiae |
| Haemophilus influenzae infection | Haemophilus influenzae |
| Hand, foot and mouth disease (HFMD) | Enteroviruses, mainly Coxsackie A virus and |
| | Enterovirus 71 (EV71) |
| Hantavirus Pulmonary Syndrome (HPS) | Sin Nombre virus |
| Helicobacter pylori infection | Helicobacter pylori |
| Hemolytic-uremic syndrome (HUS) | Escherichia coli O157:H7, O111 and O104:H4 |
| Hemorrhagic fever with renal syndrome | Bunyaviridae family |
| (HFRS) | |
| Hepatitis A | Hepatitis A Virus |
| Hepatitis B | Hepatitis B Virus |
| Hepatitis C | Hepatitis C Virus |
| Hepatitis D | Hepatitis D Virus |
| Hepatitis E | Hepatitis E Virus |
| Herpes simplex | Herpes simplex virus 1 and 2 (HSV-1 and HSV-2) |
| Histoplasmosis | Histoplasma capsulatum |
| Hookworm infection | Ancylostoma duodenale and Necator americanus |
| Human bocavirus infection | Human bocavirus (HBoV) |
| Human ewingii ehrlichiosis | Ehrlichia ewingii |
| Human granulocytic anaplasmosis (HGA) | Anaplasma phagocytophilum |
| Human metapneumovirus infection | Human metapneumovirus (hMPV) |
| Human monocytic ehrlichiosis | Ehrlichia chaffeensis |
| Human papillomavirus (HPV) infection | Human papillomavirus (HPV) |
| Human parainfluenza virus infection | Human parainfluenza viruses (HPIV) |
| Hymenolepiasis | Hymenolepis nana and Hymenolepis diminuta |
| Epstein-Barr Virus Infectious | Epstein-Barr Virus (EBV) |
| Mononucleosis (Mono) | |
| Influenza (flu) | Orthomyxoviridae family |
| Isosporiasis | Isospora belli |
| Kawasaki disease | unknown; evidence supports that it is infectious |
| Keratitis | multiple |
| Kingella kingae infection | Kingella kingae |
| Kuru | Kuru prion |
| Lassa fever | Lassa virus |
| Legionellosis (Legionnaires' disease) | Legionella pneumophila |
| Legionellosis (Pontiac fever) | Legionella pneumophila |
| Leishmaniasis | Leishmania genus |
| Leprosy | Mycobacterium leprae and Mycobacterium lepromatosis |
| Leptospirosis | Leptospira genus |
| Listeriosis | Listeria monocytogenes |
| Lyme disease (Lyme borreliosis) | usually Borrelia burgdorferi and other Borrelia species |
| Lymphatic filariasis (Elephantiasis) | Wuchereria bancrofti and Brugia malayi |
| Lymphocytic choriomeningitis | Lymphocytic choriomeningitis virus (LCMV) |
| Malaria | Plasmodium genus |
| Marburg hemorrhagic fever (MHF) | Marburg virus |
| Measles | Measles virus |
| Melioidosis (Whitmore's disease) | Burkholderia pseudomallei |
| Meningitis | multiple |
| Meningococcal disease | Neisseria meningitidis |
| Metagonimiasis | usually Metagonimus yokagawai |
| Microsporidiosis | Microsporidia phylum |
| Molluscum contagiosum (MC) | Molluscum contagiosum virus (MCV) |
| Mumps | Mumps virus |
| Murine typhus (Endemic typhus) | Rickettsia typhi |
| Mycoplasma pneumonia | Mycoplasma pneumoniae |
| Mycetoma | numerous species of bacteria (Actinomycetoma) and |
| | fungi (Eumycetoma) |
| Myiasis | parasitic dipterous fly larvae |
| Neonatal conjunctivitis (Ophthalmia | most commonly Chlamydia trachomatis and Neisseria |
| neonatorum) | gonorrhoeae |
| (New) Variant Creutzfeldt-Jakob disease (vCJD, | vCJD prion |
| nvCJD) | |
| Nocardiosis | usually Nocardia asteroides and other Nocardia species |
| Onchocerciasis (River blindness) | Onchocerca volvulus |
| Paracoccidioidomycosis (South | Paracoccidioides brasiliensis |
| American blastomycosis) | |
| Paragonimiasis | usually Paragonimus westermani and other Paragonimus species |
| Pasteurellosis | Pasteurella genus |
| Pediculosis capitis (Head lice) | Pediculus humanus capitis |
| Pediculosis corporis (Body lice) | Pediculus humanus corporis |
| Pediculosis pubis (Pubic lice, Crab lice) | Phthirus pubis |
| Pelvic inflammatory disease (PID) | multiple |
| Pertussis (Whooping cough) | Bordetella pertussis |
| Plague | Yersinia pestis |
| Pneumococcal infection | Streptococcus pneumoniae |
| Pneumocystis pneumonia (PCP) | Pneumocystis jirovecii |
| Pneumonia | multiple |
| Poliomyelitis | Poliovirus |
| Prevotella infection | Prevotella genus |
| Primary amoebic meningoencephalitis | usually Naegleria fowleri |
| (PAM) | |
| Progressive multifocal | JC virus |
| leukoencephalopathy | |
| Psittacosis | Chlamydophila psittaci |
| Q fever | Coxiella burnetii |
| Rabies | Rabies virus |
| Rat-bite fever | Streptobacillus moniliformis and Spirillum minus |
| Respiratory syncytial virus infection | Respiratory syncytial virus (RSV) |
| Rhinosporidiosis | Rhinosporidium seeberi |
| Rhinovirus infection | Rhinovirus |
| Rickettsial infection | Rickettsia genus |
| Rickettsialpox | Rickettsia akari |
| Rift Valley fever (RVF) | Rift Valley fever virus |
| Rocky mountain spotted fever (RMSF) | Rickettsia rickettsii |
| Rotavirus infection | Rotavirus |
| Rubella | Rubella virus |
| Salmonellosis | Salmonella genus |
| SARS (Severe Acute Respiratory | SARS coronavirus |
| Syndrome) | |
| Scabies | Sarcoptes scabiei |
| Schistosomiasis | Schistosoma genus |
| Sepsis | multiple |
| Shigellosis (Bacillary dysentery) | Shigella genus |
| Shingles (Herpes zoster) | Varicella zoster virus (VZV) |
| Smallpox (Variola) | Variola major or Variola minor |
| Sporotrichosis | Sporothrix schenckii |
| Staphylococcal food poisoning | Staphylococcus genus |
| Staphylococcal infection | Staphylococcus genus |
| Strongyloidiasis | Strongyloides stercoralis |
| Syphilis | Treponema pallidum |
| Taeniasis | Taenia genus |
| Tetanus (Lockjaw) | Clostridium tetani |
| Tinea barbae (Barber's itch) | usually Trichophyton genus |
| Tinea capitis (Ringworm of the Scalp) | usually Trichophyton tonsurans |
| Tinea corporis (Ringworm of the Body) | usually Trichophyton genus |
| Tinea cruris (Jock itch) | usually Epidermophyton floccosum, Trichophyton |
| | rubrum, and Trichophyton mentagrophytes |
| Tinea manuum (Ringworm of the Hand) | Trichophyton rubrum |
| Tinea nigra | usually Hortaea werneckii |
| Tinea pedis (Athlete's foot) | usually Trichophyton genus |
| Tinea unguium (Onychomycosis) | usually Trichophyton genus |
| Tinea versicolor (Pityriasis versicolor) | Malassezia genus |
| Toxocariasis (Ocular Larva Migrans | Toxocara canis or Toxocara cati |
| (OLM)) | |
| Toxocariasis (Visceral Larva Migrans | Toxocara canis or Toxocara cati |
| (VLM)) | |
| Toxoplasmosis | Toxoplasma gondii |
| Trichinellosis | Trichinella spiralis |
| Trichomoniasis | Trichomonas vaginalis |
| Trichuriasis (Whipworm infection) | Trichuris trichiura |
| Tuberculosis | usually Mycobacterium tuberculosis |
| Tularemia | Francisella tularensis |
| Ureaplasma urealyticum infection | Ureaplasma urealyticum |
| Venezuelan equine encephalitis | Venezuelan equine encephalitis virus |
| Venezuelan hemorrhagic fever | Guanarito virus |
| Viral pneumonia | multiple viruses |
| West Nile Fever | West Nile virus |
| White piedra (Tinea blanca) | Trichosporon beigelii |
| Yersinia pseudotuberculosis infection | Yersinia pseudotuberculosis |
| Yersiniosis | Yersinia enterocolitica |
| Yellow fever | Yellow fever virus |
| Zygomycosis | Mucorales order (Mucormycosis) and Entomophthorales |
| | order (Entomophthoramycosis) |

Any suitable cancer may be prevented, treated, or the prevention or treatment of the disease may be aided with the present method of inducing an immune response. In this case the antigen Ag is an antigen associated with the cancer, and the antiidiotypic antibody Ab2 binds to antibodies Ab1 specific for a cancer antigen Ag. Examples of identified or suspected cancers include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) Cancer; Gastric (Stomach) Cancer, Childhood; Gastrointestinal Carcinoid Tumor; Gastrointestinal Stromal Tumor (GIST); Germ Cell Tumor, Extracranial, Childhood; Germ Cell Tumor, Extragonadal; Germ Cell Tumor, Ovarian; Gestational Trophoblastic Tumor; Glioma, Adult; Glioma, Childhood Brain Stem; Glioma, Childhood Cerebral Astrocytoma; Glioma, Childhood Visual Pathway and Hypothalamic; Gastric Carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Macroglobulinemia, Waldenström; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sézary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sézary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenström macroglobulinemia; and Wilms tumor (kidney cancer), childhood.

Certain cancers are caused by infectious pathogens, and in such cases the cancers may be prevented by immunization against the infectious pathogen using the present method.

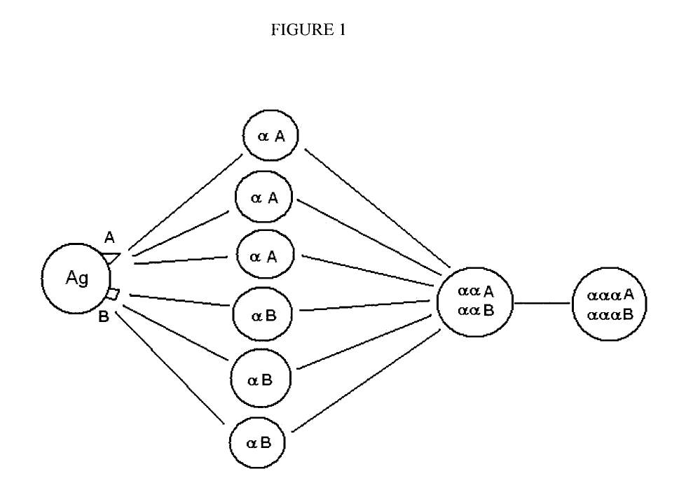

FIG. 1 shows a model of how the immune system reacts to an antigen Ag with two antigenic features A and B. The symbol a is an abbreviation for “anti-”. The interactions between clones are symmetrical. The αA and αB clones are stimulated by ααA/ααB clones and vice versa. The clones with receptors that have affinity for the antigen are diverse, and there is a process called co-selection involving firstly αA and αB clones and secondly ααA/ααB clones. G. W. Hoffmann (1994) Cell Biol. 72, 338. Clones that are only ααA or only ααB are not selected nearly as strongly as those that are both ααA and ααB. The criterion for the selection of ααA/ααB clones is that they recognize as many αA and αB clones as possible. This criterion together with the diversity of the αA and αB clones and the non-linear autocatalytic co-selection process results in the selection of the homogeneous antiidiotypic clones ααA/ααB. This homogeneous population emerges as a stronger “antigen” than Ag, and stimulates αααA/αααB clones. Any clone that has complementarity to the ααA/ααB clones is αααA/αααB, so the nomenclature becomes ambiguous, with αααA/αααB including αA and αB clones and clones that have complementarity to ααA/ααB but being neither αA nor αB. The mutual selection of (a) diverse αA and αB clones and (b) less diverse ααA/ααB clone or clones sharply defines a polarization of the network that is associated with the antigen. The αααA/αααB clones which are neither αA nor αB are generalizations of the Ag pattern in the context of the network. This model can be generalized to include the antigen having more than two features. The model explains the phenomenon that Jerne called the “Oudin-Cazenave enigma”, namely the remarkable finding that an immune response to an antigen can include antibodies to different epitopes of an antigen that express the same idiotype, and can also include antibodies that do not bind to the antigen at all. J. Oudin et al., (1971) Proc. Nat. Acad. Sci. (USA) 68, 2616; N. K. Jerne (1974) Ann. Immunol. (Inst. Pasteur), 125C, 373.

While not wishing to be bound by theory, it is suggested that immune responses may involve the selection of a small diversity of antiidiotypic clones, which may be a single clone or a family of closely related clones. The V regions of these clones may be powerful antigens that play a central role in the immune response to the antigen.

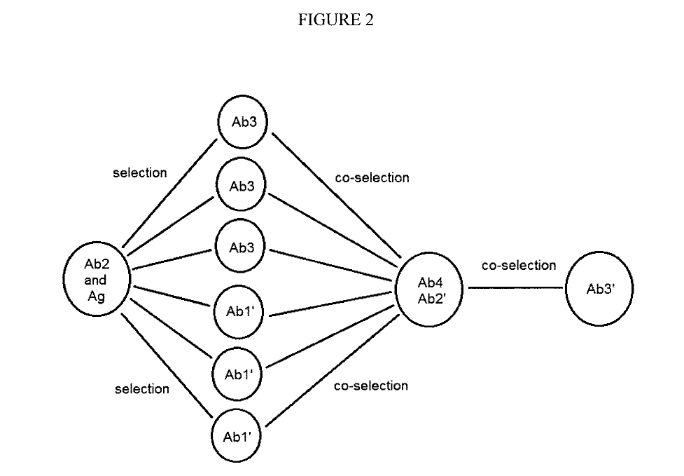

While not wishing to be bound by theory, FIG. 2 shows a proposed mechanism of how the present vaccines may work. Here Ab2 is an antiidiotypic antibody that binds to antibodies Ab1 that bind to an antigen or antigens Ag of a pathogen or of a cancer. Ab2 stimulates Ab3 clones and Ag stimulates Ab1′ clones. There is co-selection of (a) some of the Ab3 clones and (b) some of the Ab1′ clones with (c) those lymphocytes that are both Ab4 and Ab2′. The use of the “Ab” nomenclature is not meant to imply that all these populations are all B cells. On the contrary, a homogeneous co-selected population may comprise T cells that are stabilized by a heterogeneous population of B cells. If the Ab4/Ab2′ population emerges as the strongest antigen in the system, it is believed to be a homogeneous population of Ab4/Ab2′ T cells that is stabilized by a heterogeneous population of Ab3′ B cells. If the Ab3′ population emerges as the strongest antigen in the system, it is believed to be a homogeneous population of Ab3′ T cells, which is stabilized by a heterogeneous population of Ab4/Ab2′ B cells.

The present disclosure provides, in part, a vaccine against an infectious pathogen comprising complexes and/or mixtures of (a) antiidiotypic antibodies Ab2 specific for antibodies Ab1 that are specific for pathogen antigens Ag and (b) one or more pathogen antigens. The present disclosure also provides a vaccine comprising complexes and/or mixtures of (a) antiidiotypic antibodies Ab2 that are specific for antibodies Ab1 that are specific for a cancer and (b) an antigen or antigens Ag of the cancer.

Suitable monoclonal antiidiotypic antibodies for use herein may be obtained by immunization of a vertebrate with antibodies Ab1 specific for the pathogen or cancer and selection of monoclonal antibodies that preferably bind to multiple antibodies specific for epitopes on the pathogen or cancer.

As a vaccine the complexes or mixtures of the antiidiotypic antibody Ab2 and the antigen Ag may be given in an immunogenic dose and/or in an immunogenic form, that is, an immunogenic dose with an adjuvant. An effective immunogenic dose may, for example, be in the range of 10 μg to 1 mg of the complexes or mixtures. The vaccine may be given one, two or more times as needed to induce the desired result such as protection against the infectious agent or cancer.

While not wishing to be bound by theory, prior to immunization with Ab2/Ag complexes, there is believed to be a symmetry between the Ab4/Ab2′ lymphocytes and the Ab3′ lymphocytes. There are as many Ab4/Ab2′ lymphocytes as Ab3′ lymphocytes. It is believed the symmetry is broken in the course of an immune response. There are two kinds of response. One of the two populations is destined to become the strongest antigen in the system, namely either Ab4/Ab2′ or Ab3′. It is believed that in each case the strongest antigen is a homogeneous population of T cells, while the B cell response is diverse and includes proliferation and the production of antibodies. If Ab4/Ab2′ T cells become the strongest antigen, the strongest antibody response will be that of Ab3′ B cells. We call this a type 1 response. The Ab3′ antibodies include antibodies that are specific for Ab2 and/or Ag. On the other hand, if Ab3′ T cells become the strongest antigen, the strongest antibody response is that of the Ab4/Ab2′ B cells. We call this a type 2 response. This response includes the production of antibodies that bind to an Ab3 molecule, for example bind to a BnAb, since BnAbs are assumed to be Ab3 antibodies. G. W. Hoffmann, S. Muller and H. Kohler (2012) Curr. Trends Immunol. 13, 69-79 (incorporated herein by reference). We may therefore determine whether there has been a type 1 response or a type 2 response according to whether there is a stronger antibody response to Ag and/or to Ab2 than to a BnAb (type 1 response), or a stronger antibody response to a BnAb than to Ag and/or to Ab2 (type 2 response).

The present disclosure also provides for the induction of an immune response using a mixture of an antiidiotypic antibody and a BnAb. In this case we again determine whether there has been type 1 or a type 2 immune response by determining whether there has been a stronger antibody response to Ag and/or Ab2 than to a BnAb (type 1) or a stronger antibody response to a BnAb than the antibody response to the antigen Ag and/or Ab2 (type 2).

The present disclosure also provides for the induction of an immune response using a complex or a mixture of (a) an antibody and (b) a pathogen or cancer antigen Ag. The antibody may for example be an IgM antibody or an IgG antibody. In this case we again determine whether there has been type 1 or a type 2 immune response by determining whether there has been a stronger antibody response to Ag and/or Ab2 than to a BnAb (type 1) or a stronger response to a BnAb than the antibody response to the antigen Ag and/or Ab2 (type 2).

The immune response may be boosted by immunizing with an antigen that stimulates the strongest antigen in the system. For the case of a type 1 response the strongest antigen is Ab4/Ab2′ T cells, and the vertebrate may be boosted by immunizing with a BnAb, that is, an Ab3. For the case of a type 2 response, with Ab3′ being the strongest antigen in the system, the response may be boosted by immunizing with an antigen that is complementary to a BnAb, for example Ab2 or the antigen Ag.

A fragment of an antibody, for example a Fab molecule, may substitute for an antibody.

Monoclonal antiidiotypic antibodies suitable for use in this invention include the monoclonal anti-anti-HIV antibody 1F7. Wang et al. (1992) Eur. J. Immunol. 22, 1749, herein incorporated by reference. 1F7 binds to anti-HIV antibodies in about 73% of HIV-1 positive sera. 1F7 furthermore binds to six well-characterized monoclonal broadly neutralizing anti-HIV antibodies, namely 812, 2G12, VRC01, 2F5, 4E10 and Z13. Parsons et al. (2011) AIDS 25, 1259, herein incorporated by reference. The mAb1F7 also binds to antibodies present in macaque monkeys infected with simian immunodeficiency virus (SIV). The fact that 1F7 binds to all of six well-characterized monoclonal broadly neutralizing anti-HIV antibodies is consistent with the concept that monoclonal broadly neutralizing anti-HIV antibodies are Ab3 antibodies. Hoffmann et al. (2012), op cit. 1F7 binds also to antibodies specific for hepatitis C virus (HCV). M. D. Grant (2002) J. Med. Virol. 66, 13; T. K. Davtyan et al. (2009) Immunol. Cell Biol. 87, 457, all herein incorporated by reference. Humans who become infected with HCV typically become chronically infected, which in this regard is similar to infection with HIV. Hence a vaccine comprising 1F7 plus a part of an infectious agent that causes chronic infection, for example HCV, will plausibly work as a vaccine also against said infectious agent.

Monoclonal broadly neutralizing anti-HIV antibodies suitable for use in this invention include B12, 2G12, VRC01, 2F5, 4E10 and Z13.

An antiidiotypic antibody Ab2 and an antigen Ag may be mixed and complexes may be formed that contain Ab2 and Ag. This can be done for example using a cross-linking reagent, for example MBS (m-maleimidobenzoyl-N-hydoxysuccinimide ester), DSS (disuccinimidyl suberate), BS³ (bis[sulfosuccinimidyl] suberate), glutaraldehyde, adipimidate, dimethyl suberimidate or dimethyl pimelimidate. Complexes may also be formed for example by other methods that cause mild denaturation of Ab2 and Ag, for example heat shock, change in pH or change in ionic strength.

The present disclosure provides a method for inducing an immune response in a vertebrate “A” to an antigen Ag comprising: obtaining Ag-specific antibodies, for example, from a vertebrate “B” that has been immunized with Ag; producing monoclonal antiidiotypic antibodies specific for the said Ag-specific antibodies; producing complexes and/or mixtures of (a) said monoclonal antiidiotypic antibodies and (b) the antigen Ag; immunizing the vertebrate A with the said complexes or mixtures. The present antiidiotypic antibody may bind to multiple antibodies with specificity for multiple epitopes of the antigen Ag

The present disclosure provides a method for immunizing a vertebrate “A” against infection with a pathogen comprising: obtaining pathogen-specific antibodies, for example from a vertebrate “B” that has been immunized with or infected by the pathogen, or immunized with pathogen antigen or antigens; producing monoclonal antiidiotypic antibodies specific for the said pathogen-specific antibodies; producing complexes and/or mixtures of (a) said monoclonal antiidiotypic antibodies and (b) the corresponding pathogen antigen or antigens; immunizing the vertebrate A with the said complexes or mixtures.

A method for the treatment or prevention of a cancer, for the case that there are one or more antigens associated with the cancer, said method comprising: obtaining cancer antigen-specific antibodies, for example from a vertebrate that has been immunized with the cancer antigen-specific antigen or antigens; producing monoclonal antiidiotypic antibodies specific for the cancer-specific antibodies; producing complexes and/or mixtures of (i) monoclonal antiidiotypic antibodies and (ii) the corresponding cancer antigen or antigens; immunizing a vertebrate with the said complexes and/or mixtures.

The present infectious agent may be HIV and the monoclonal antiidiotypic antibody may be 1F7.

The pathogen antigen may be one or more of the proteins or glycoproteins of HIV or SIV, or fragments thereof.

The present disclosure provides a kit comprising: complexes and/or mixtures of a monoclonal antiidiotypic antibody and the corresponding antigen and a pharmaceutically acceptable carrier; and instructions for use.

The present disclosure provides a pharmaceutical composition comprising a monoclonal antiidiotypic antibody complexed to and/or mixed with the corresponding antigen, and a pharmaceutically acceptable carrier. The pharmaceutically acceptable carrier may comprise any suitable material. Preferably the pharmaceutically acceptable carrier comprises an adjuvant.

The present disclosure provides the use of a monoclonal antiidiotypic antibody complexed to and/or mixed with the corresponding antigen as a therapeutic or preventive vaccine against an infectious agent or cancer.

The present disclosure provides a method for immunizing a vertebrate “A” against an infectious agent that causes chronic infection comprising: producing complexes and/or mixtures of (i) 1F7 and (ii) an antigen of the infectious agent; and immunizing the vertebrate A with the said complexes or mixtures.

A method for inducing an immune response in a vertebrate “A” to an antigen Ag comprising: obtaining Ag-specific antibodies from a vertebrate “B” that has been immunized with Ag; producing monoclonal antiidiotypic antibodies specific for the said Ag-specific antibodies; producing complexes and/or mixtures of (i) said monoclonal antiidiotypic antibodies and (ii) the antigen Ag; immunizing the vertebrate A with the said complexes and/or mixtures; and immunizing the vertebrate with a broadly neutralizing monoclonal anti-HIV antibody.

The present disclosure provides a method for inducing an immune response in a vertebrate “A” to an antigen Ag comprising: obtaining Ag-specific antibodies from a vertebrate “B” that has been immunized with Ag; producing monoclonal antiidiotypic antibodies specific for the said Ag-specific antibodies; producing a two-component vaccine comprising complexes and/or mixtures of (i) said monoclonal antiidiotypic antibodies and (ii) the antigen Ag; immunizing the vertebrate A with the said complexes and/or mixtures; and immunizing the vertebrate with one of the components of the two-component vaccine.

The present disclosure provides a method for inducing an immune response in a vertebrate “A” to an antigen Ag comprising: obtaining Ag-specific antibodies from a vertebrate “B” that has been immunized with Ag; producing monoclonal antiidiotypic antibodies specific for the said Ag-specific antibodies; producing mixtures and/or complexes of (i) said monoclonal antiidiotypic antibodies and (ii) a monoclonal broadly neutralizing anti-HIV antibody; immunizing the vertebrate A with the said complexes and/or mixtures; and immunizing the vertebrate with a broadly neutralizing monoclonal anti-HIV antibody.

The present disclosure provides a method for inducing an immune response in a vertebrate “A” to an antigen Ag comprising: obtaining Ag-specific antibodies from a vertebrate “B” that has been immunized with Ag; producing monoclonal antiidiotypic antibodies specific for the said Ag-specific antibodies; producing mixtures and/or complexes of (i) said monoclonal antiidiotypic antibodies and (ii) a monoclonal broadly neutralizing anti-HIV antibody; immunizing the vertebrate A with the said mixtures and/or complexes; and immunizing the vertebrate with the monoclonal antiidiotypic antibody.

The present disclosure provides a method for inducing an immune response in a vertebrate “A” to an antigen Ag comprising: producing complexes and/or mixtures of (i) an antibody Ab and (ii) the antigen Ag; immunizing the vertebrate A with the said complexes and/or mixtures; and immunizing the vertebrate with a broadly neutralizing monoclonal anti-HIV The present disclosure provides method for inducing an immune response in a vertebrate “A” to an antigen Ag comprising: producing complexes and/or mixtures of (i) an antibody Ab and (ii) the antigen Ag; immunizing the vertebrate A with the said complexes and/or mixtures; and immunizing the vertebrate with the antigen Ag or the antibody Ab. The method of claim 18 or claim 19, whereby the antibody Ab is an IgM molecule. The antibody Ab may, for example, be an IgG molecule.

The present antigen Ag may, for example, be a pathogen antigen, a cancer antigen, or a mixture of antigens.

The present disclosure provides a kit comprising: a two-component vaccine and a pharmaceutically acceptable carrier; a monoclonal broadly neutralizing anti-HIV antibody and a pharmaceutically acceptable carrier; one of the two components of said two-component vaccine and a pharmaceutically acceptable carrier; and instructions for use.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

It is contemplated that any embodiment discussed in this specification can be implemented or combined with respect to any other embodiment, method, composition or aspect of the invention, and vice versa.

### Examples

In an experiment involving 10 rabbits, the rabbits are immunized on days 0, 14 and 28, and are bled on days 0, 7, 14, 21, 28, 35 and 42. Rabbits number 1 and 2 are immunized each time with 100 μg of 1F7-gp120 complexes with alum. Rabbits 3 and 4 are immunized each time with 100 μg of 1F7-MPER complexes with alum, where MPER is the membrane-proximal ectodomain region of HIV-1 gp41. Rabbit 5 is immunized on day 0 with approximately 50 μg gp120 in Complete Freund's Adjuvant (CFA), and on days 14 and 28 with approximately 50 μg gp120 in incomplete Freund's adjuvant (ICFA). Rabbit 6 is immunized each time with a mixture of 50 μg of 1F7 and 50 μg of the BnAb B12 with alum. Rabbits 7 and 8 are immunized each time with 1F7 that is sham treated, that is the step of forming complexes with gp120 or MPER are taken, but in the absence of gp120 or MPER. These immunizations are also with alum. 1F7 is an IgM/κ monoclonal antibody and rabbits 9 and 10 are immunized each time with complexes of the IgM/κ monoclonal antibody TEPC183 and MPER with alum.

### Materials and Methods

### Antigens

Recombinant HIV-1 gp120-IIIB and rgp41-MN are purchased from Immunodiagnostics, Inc., Woburn, Mass. B12, a recombinant human monoclonal antibody to HIV-1 gp120, blocking binding to CD4, is purchased from PolyMun, Klosterneuburg, Austria. Gp41-MPER peptide with the amino acid sequence CELLELDKWASLWNWFDITNWLWYIK is synthesized by Genemed Synthesis, San Antonio, Tex. Mouse monoclonal (mAb) 1F7, IgM, kappa is provided by Drs. Heinz Kohler and Sybille Muller, Immpheron, Inc., Lexington, Ky. (Muller et al., 1991, Wang et al, 1992). TEPC183 (IgM, kappa murine myeloma) is obtained from Sigma Aldrich, St. Louis, Mo. Goat polyclonal anti-HIV-1 gp41 antibody-HRP is purchased from Abcam Inc., Cambridge, Mass.

### Conjugation

5 mg 1F7 PBS pH 7.2 are dissolved in 10M excess MBS (m-maleimidobenzoyl-N-hydoxysuccinimide ester) 30 fold excess peptide or rgp120 is added. Dissolve 20 mg MBS in 1 ml DMF. Immediately add 60 ul of this solution to antibody (5 mg/ml). React at room temperature for 30 min on lab quake. Desalt over PD-10 column. Add 30 fold excess MPER cys peptide and react for 30 min at room temperature. Add 50 ul of 100 mM cys to quench the reaction and react 30 min at room temperature. Desalt over PD-10 column (Protocol is provided by Thermo Scientific, Pierce Biotechnology, Rockford, Il)

### Vaccination

Rabbits are injected using 1F7 MBS Conjugation with Cys linker. Total of 6 groups are enrolled. Group 1 (n=2) received 1F7-gp120 conjugate in alum; Group 2 (n=2) received 1F7-MPER conjugate in alum; Group 3 (n=1) received gp120 in complete Freund's adjuvant; Group 4 (n=1) received 1F7 plus B12 in alum. Similarly, Group 5 (n=2) received 1F7 cross-linked sham-treated in alum and Group 6 (n=2) received Mouse IgM-peptide conjugate in alum, whereby the IgM is TEPC183.

### Administration of Antigens

All animals are immunized with the antigens on days 0, 14 and 28. Group 1 animals (Rabbits R1 and R2) received 100 ug 1F7-gp120 conjugate with alum. Group 2 animals (Rabbits R3 and R4) received 100 μg 1F7-MPER conjugate with alum. Group 3 animal (Rabbit R5) received approximately 50 μs gp120 with Freund's complete adjuvant on day 0, followed by approximately 50 μg gp120 in Freund's incomplete adjuvant on days 14 and 28. Group 4 animal (Rabbit R6) received 50 μg 1F7 plus 50 μg B12. Group 5 animals (Rabbits R7 and R8) received 100 μg 1F7 cross-linked sham-treated with alum. Group 6 animals (Rabbits R9 and R10) received 100 μg Mouse IgM-peptide conjugate with alum.

### ELISA Assays

Rabbit serum antibodies binding to HIV-1 antigens is tested by ELISA. ELISA is performed as previously described (S. Muller et al. 1991, H. Wang et al. 1992). ELISAs are performed using sera at Day 0 (pre-bleed) and Day 35, and 42, respectively, after vaccination. Then sera from day 7, 14, 35, and 42 after vaccination of selected rabbits are tested.

### ELISA 1

Microtiter plate wells are coated with 100 ng of either gp120 or gp41 incubate overnight at 4° C. and then the wells are washed with 1× with PBS 0.05% tween 20 (ELISA wash buffer). ELISA Wash buffer for gp41 assay contained 0.1% tween 20. The plate wells are blocked with Blotto (5% milk powder) 1 hr at 37° C., and washed with one time ELISA wash buffer. 10 ul of serum per 190 ul PBS are added and serially diluted. The plates are incubated at 4° C. overnight. The wells are washed three times with ELISA wash buffer. 1:5000 of goat anti-rabbit IgG HRP are added, and incubated 1 hour at room temperature, then washed 3× with ELISA wash buffer. Color is developed with 100 ul OPD for 6 minutes and then stopped with 50 ul 2N H2SO4. Absorption of plate wells are read in a spectrophotometer at OD492.

ELISA 2

To show antigenicity (gp41-MPER) of the gp41-MPER-peptide conjugated to 1F7 antibody, plate wells are coated with 200 ng gp41-MPER-1F7 conjugate, blocked and washed as described above. Then goat polyclonal anti-HIV-1 gp41 antibody-HRP is added, and binding determined and visualized as described above in ELISA 1. Significant binding could be detected (data not shown).

### Figures

Mean and standard error of the mean is calculated from duplicate OD readings, and plotted by Prism analysis program and the graphs exported to Microsoft Powerpoint.

### REFERENCES

Partis, M. D., et al. (1983). Cross-linking of protein by [symbol] -maleimido alkanoyl N-hydroxysuccinimido esters. J Prot Chem 2(3):263-77.
Myers, D. E., et al. (1989). The effects of aromatic and aliphatic maleimide crosslinkers on anti-CD5 ricin immunotoxins. J Immunol Meth 121:129-42.
Russ, M., Lou, D., Kohler, H. (2005). Photo-activated affinity-site cross-linking of antibodies using tryptophan containing peptides, Jour Imm Meth 304, 100-106.
Muller, S., et al., (1991). Generation and specificity of monoclonal anti-idiotypic antibodies against human HIV-specific antibodies. J Immunol 147: 933-941.
Wang, H., et al, (1992). Human monoclonal and polyclonal anti-human immunodeficiency virus-1 antibodies share a common clonotypic specificity. Eur J Immunol 22: 1749-1755.

### Results

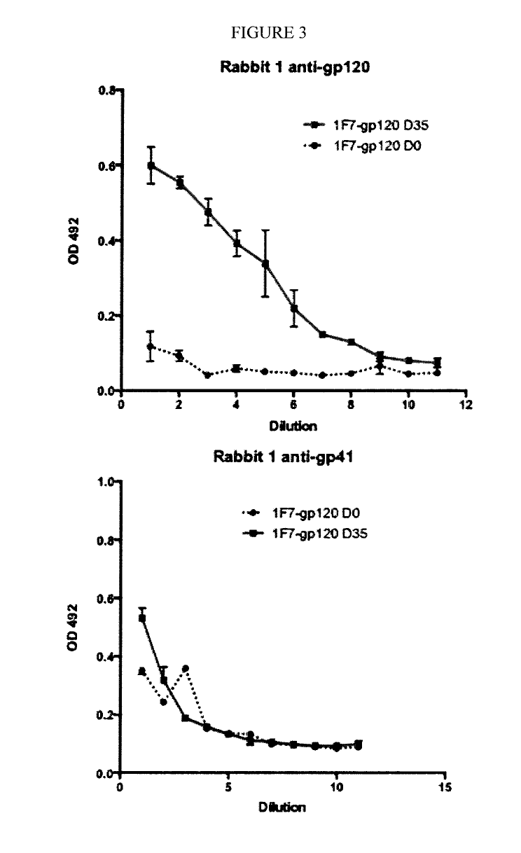

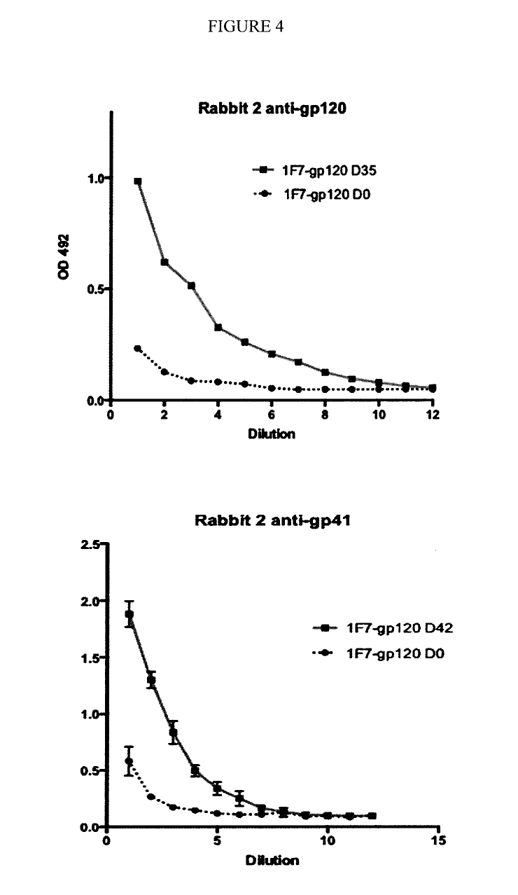

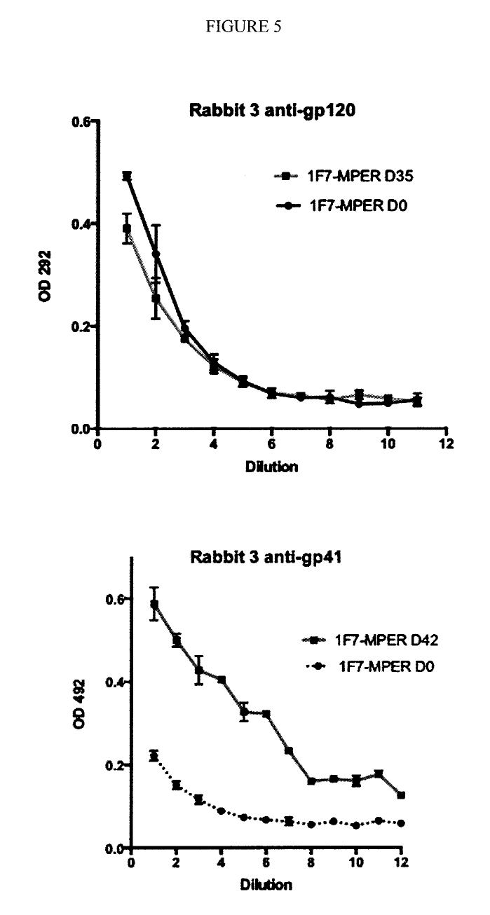

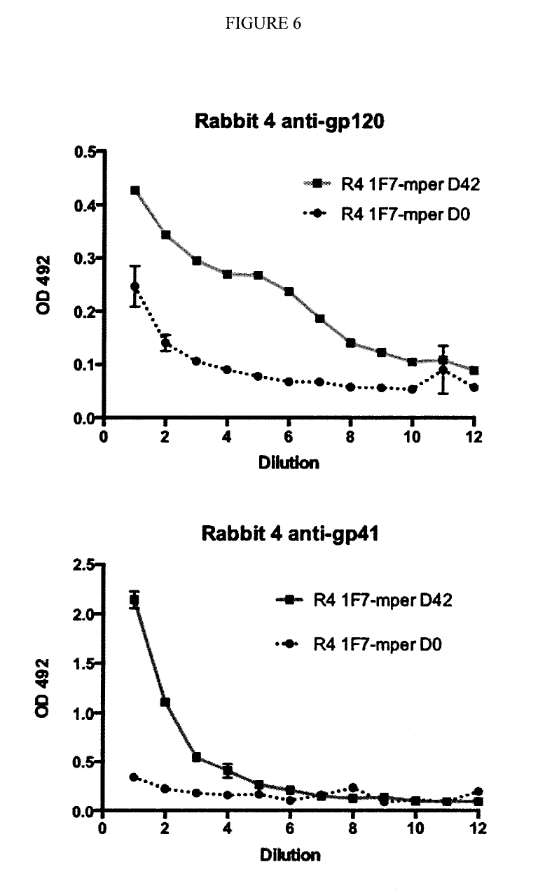

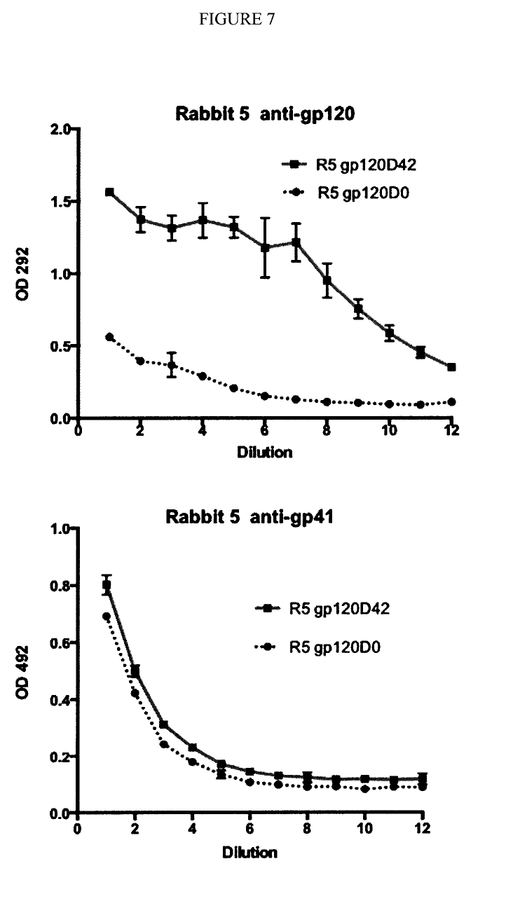

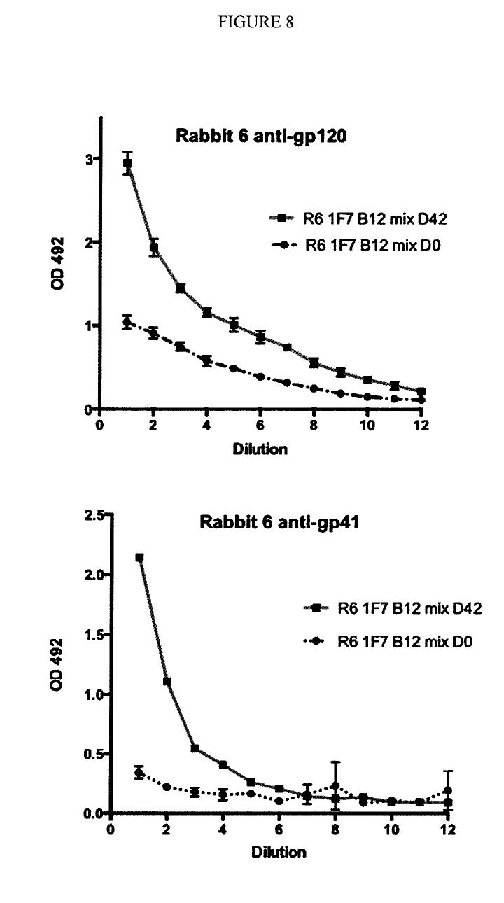

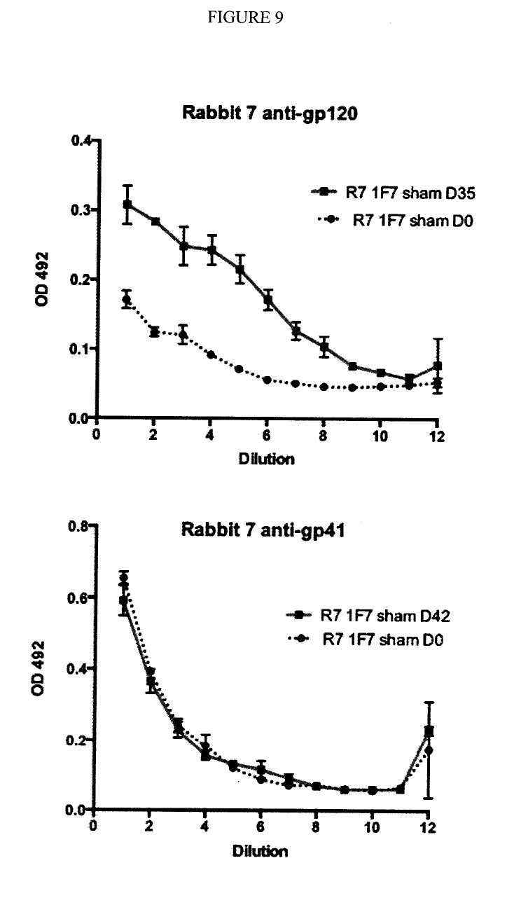

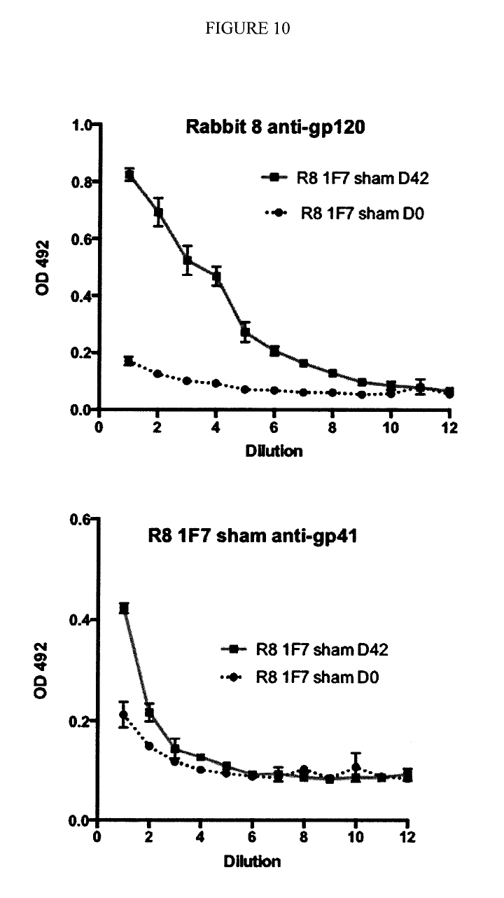

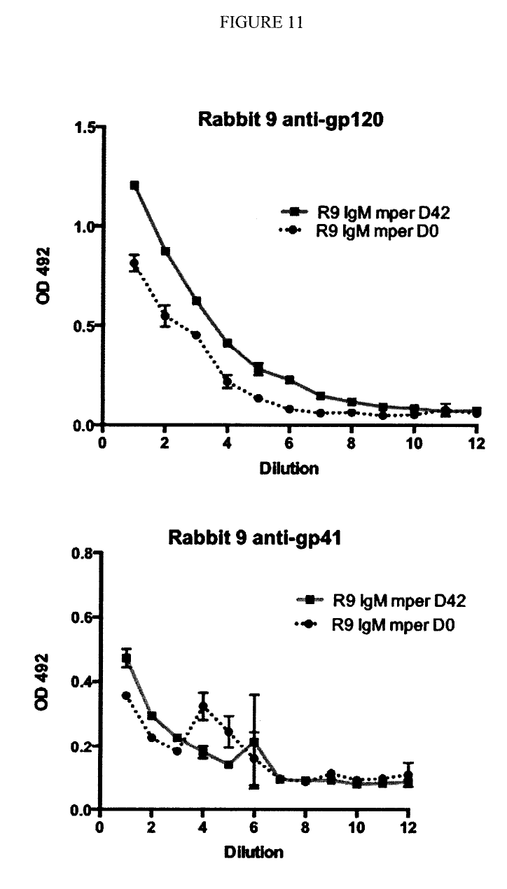

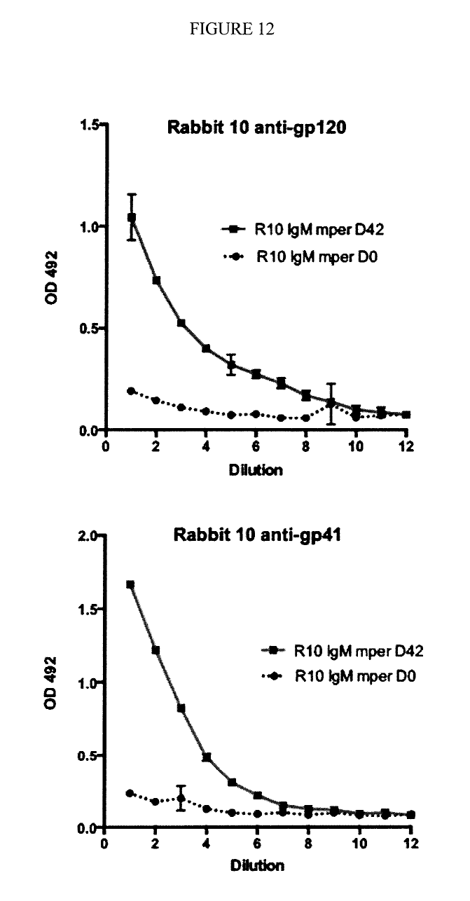

ELISA results for gp120 binding and gp41 binding antibodies in serum samples taken from the ten rabbits at day 0 and day 42 are shown in FIGS. 3 to 12. One of the two rabbits immunized with 1F7-gp120 complexes (rabbit 2) responded with the production of both anti-gp120 binding and gp41 binding antibodies, even though the immunogen contained gp120 and no gp41. In this sense the response to 1F7-gp120 is broad. Similarly, one of the two rabbits immunized with 1F7-MPER (rabbit 4) responded with the production of both anti-gp120 and anti-gp41 antibodies. MPER is a gp41 peptide, so the response to gp120 is evidence of a broad response. The control rabbit 5, that is immunized with gp120 in CFA and then twice with gp120 in ICFA, responded with the production of a high titre of gp120 binding antibodies and no gp41 binding antibodies as expected. Rabbit 6 responded to the mixture of 1F7 and B12 with the production of both anti-gp120 and anti-gp41 antibodies, which is evidence of a broad response in the absence of either gp120 or gp41 as part of the immunogen. The control rabbits 7 and 8 both responded to immunization with sham treated 1F7 with the production of gp120 binding antibodies, while there is a weak response (rabbit 8) or no response (rabbit 7) to gp41. Of the control rabbits 9 and 10 that are immunized with complexes of the monoclonal antibody TEPC183 and MPER, in rabbit 9 there is a high background level of anti-gp120 antibodies at day 0 which increased following immunization, and no gp41 binding antibodies, while rabbit 10 responded to immunization with the production of both gp120 binding and gp41 binding antibodies. Hence MPER coupled to an IgM antibody is able to induce a broad response.

It is notable that one of the two rabbits 1 and 2 responded by making both anti-gp120 and anti-gp41 antibodies, one of the two rabbits 3 and 4 responded by making both anti-gp120 and anti-gp41 antibodies, and one of the two rabbits 9 and 10 responded by making both anti-gp120 and anti-gp41 antibodies. This is consistent with the existence of two kinds of response for stimulation with the corresponding antigens. For example, in the case of rabbits 1 and 2, in addition to the possibility of stimulation with complexes of 1F7 and gp120 leading to Ab4/Ab2′ T cells becoming the strongest antigen in the system, causing stimulation of Ab3′ B cells (rabbit 2), Ab3′ T cells may emerge as the strongest antigen in the system, resulting in stimulation of Ab4/Ab2′ B cells (rabbit 1). Similarly, in the case of rabbits 3 and 4, in addition to the possibility of stimulation with complexes of 1F7 and MPER leading to Ab4/Ab2′ becoming the strongest antigen in the system, with stimulation of Ab3′ B cells (rabbit 4), Ab3′ may emerge as the strongest antigen in the system, resulting in stimulation of Ab4/Ab2′ B cells (rabbit 3).

It is anticipated that a stronger response could be induced in rabbits 2, 4, 6 and 10 by boosting with a BnAb, while a stronger response could be induced in rabbits 1, 3 and 9 by boosting with one of the components of the two-component vaccine.

1. A method for inducing an immune response in a vertebrate “A” to an antigen Ag comprising:
a) obtaining Ag-specific antibodies;
b) producing monoclonal antiidiotypic antibodies specific for the said Ag-specific antibodies;
c) producing complexes and/or mixtures of (a) said monoclonal antiidiotypic antibodies and (b) the antigen Ag;
d) immunizing the vertebrate A with the said complexes or mixtures.

2. A method for immunizing a vertebrate “A” against infection with a pathogen comprising:
a) obtaining pathogen-specific antibodies from a vertebrate “B” that has been immunized with or infected by the pathogen, or immunized with pathogen antigen or antigens;
b) producing monoclonal antiidiotypic antibodies specific for the said pathogen-specific antibodies;
c) producing complexes and/or mixtures of (a) said monoclonal antiidiotypic antibodies and (b) the corresponding pathogen antigen or antigens;
d) immunizing the vertebrate A with the said complexes or mixtures.

3. A method for the treatment or prevention of a cancer, said method comprising:
a) obtaining cancer antigen-specific antibodies from a vertebrate that has been immunized with the cancer antigen-specific antigen or antigens;
b) producing monoclonal antiidiotypic antibodies specific for the cancer-specific antibodies;
c) producing complexes and/or mixtures of (i) monoclonal antiidiotypic antibodies and (ii) the corresponding cancer antigen or antigens;
d) immunizing a vertebrate with the said complexes and/or mixtures.

4. The method of claim 2 whereby the infectious agent is HIV and the monoclonal antiidiotypic antibody is 1F7.

5. The method of claim 2 or 4 whereby the pathogen antigen is one or more of the proteins or glycoproteins of HIV or SIV, or fragments thereof.

6. A kit comprising:
a) complexes and/or mixtures of a monoclonal antiidiotypic antibody and the corresponding antigen and a pharmaceutically acceptable carrier; and
b) instructions for use.

7. A pharmaceutical composition comprising a monoclonal antiidiotypic antibody complexed to and/or mixed with the corresponding antigen, and a pharmaceutically acceptable carrier.

8. Use of a monoclonal antiidiotypic antibody complexed to and/or mixed with the corresponding antigen as a vaccine against an infectious agent.

9. Use of a monoclonal antiidiotypic antibody complexed to and/or mixed with the corresponding antigen as a therapeutic vaccine against a cancer.

10. Use of a monoclonal antiidiotypic antibody complexed to and/or mixed with the corresponding antigen as a preventive vaccine against a cancer.

11. The method of claim 1 whereby the antiidiotypic antibody binds to multiple antibodies with specificity for multiple epitopes of the antigen Ag.

12. A method for immunizing a vertebrate “A” against an infectious agent that causes chronic infection comprising:
a) producing complexes and/or mixtures of (i) 1F7 and (ii) an antigen of the infectious agent; and
b) immunizing the vertebrate A with the said complexes or mixtures.

13. The pharmaceutical composition of claim 7, whereby the pharmaceutically acceptable carrier comprises an adjuvant.

14. A method for inducing an immune response in a vertebrate “A” to an antigen Ag comprising:
a) obtaining Ag-specific antibodies from a vertebrate “B” that has been immunized with Ag;
b) producing monoclonal antiidiotypic antibodies specific for the said Ag-specific antibodies;
c) producing complexes and/or mixtures of (i) said monoclonal antiidiotypic antibodies and (ii) the antigen Ag;
d) immunizing the vertebrate A with the said complexes and/or mixtures; and
e) immunizing the vertebrate with a broadly neutralizing monoclonal anti-HIV antibody.

15. A method for inducing an immune response in a vertebrate “A” to an antigen Ag comprising:
a) obtaining Ag-specific antibodies from a vertebrate “B” that has been immunized with Ag;
b) producing monoclonal antiidiotypic antibodies specific for the said Ag-specific antibodies;
c) producing a two-component vaccine comprising complexes and/or mixtures of (i) said monoclonal antiidiotypic antibodies and (ii) the antigen Ag;
d) immunizing the vertebrate A with the said complexes and/or mixtures; and
e) immunizing the vertebrate with one of the components of the two-component vaccine.

16. A method for inducing an immune response in a vertebrate “A” to an antigen Ag comprising:
a) obtaining Ag-specific antibodies from a vertebrate “B” that has been immunized with Ag;
b) producing monoclonal antiidiotypic antibodies specific for the said Ag-specific antibodies;
c) producing mixtures and/or complexes of (i) said monoclonal antiidiotypic antibodies and (ii) a monoclonal broadly neutralizing anti-HIV antibody;
d) immunizing the vertebrate A with the said complexes and/or mixtures; and
e) immunizing the vertebrate with a broadly neutralizing monoclonal anti-HIV antibody.

17. A method for inducing an immune response in a vertebrate “A” to an antigen Ag comprising:
a) obtaining Ag-specific antibodies from a vertebrate “B” that has been immunized with Ag;
b) producing monoclonal antiidiotypic antibodies specific for the said Ag-specific antibodies;
c) producing mixtures and/or complexes of (i) said monoclonal antiidiotypic antibodies and (ii) a monoclonal broadly neutralizing anti-HIV antibody;
d) immunizing the vertebrate A with the said mixtures and/or complexes; and
e) immunizing the vertebrate with the monoclonal antiidiotypic antibody.

18. A method for inducing an immune response in a vertebrate “A” to an antigen Ag comprising:
a) producing complexes and/or mixtures of (i) an antibody Ab and (ii) the antigen Ag;
b) immunizing the vertebrate A with the said complexes and/or mixtures; and
c) immunizing the vertebrate with a broadly neutralizing monoclonal anti-HIV

19. A method for inducing an immune response in a vertebrate “A” to an antigen Ag comprising:
a) producing complexes and/or mixtures of (i) an antibody Ab and (ii) the antigen Ag;
b) immunizing the vertebrate A with the said complexes and/or mixtures; and
c) immunizing the vertebrate with the antigen Ag or the antibody Ab.

20. The method of claim 18 or claim 19, whereby the antibody Ab is an IgM molecule.

21. The method of claim 18 or claim 19, whereby the antibody Ab is an IgG molecule.

22. A method for immunizing a vertebrate “A” against infection with a pathogen comprising the immunization method of claim 14, 15, 16, 17, 18, 19, 20 or 21 whereby the antigen Ag is a pathogen antigen.

23. A method for preventing or treating a cancer comprising the immunization method of claim 14, 15, 16, 17, 18, 19, 20, or 21 whereby the antigen Ag is a cancer antigen.

24. A method for immunizing a vertebrate “A” against infection with a pathogen comprising the immunization method of claim 14, 15, 16 or 17 whereby the infectious agent is HIV and the monoclonal antiidiotypic antibody is 1F7.

25. The method of claim 22 or 24 whereby the pathogen antigen is one or more of the proteins or glycoproteins of HIV or SIV, or fragments thereof.

26. A kit comprising:
a) a two-component vaccine and a pharmaceutically acceptable carrier;
b) a monoclonal broadly neutralizing anti-HIV antibody and a pharmaceutically acceptable carrier;
c) one of the two components of said two-component vaccine and a pharmaceutically acceptable carrier; and
d) instructions for use.
